(19) United States
(12) Patent Application Publication
(10) Pub. No.: US 2019/0099612 A1
(43) Pub. Date: Apr. 4, 2019

(54) ENDOSCOPE

(71) Applicant: Dornier MedTech Laser GmbH, Wessling (DE)

(72) Inventors: Werner Hiereth, Gilching (DE); Benjamin Bergmann, Kottgeisering (DE); Michael Rampp, Munchen (DE)

(21) Appl. No.: 16/141,586

(22) Filed: Sep. 25, 2018

(30) Foreign Application Priority Data
Sep. 25, 2017 (DE) 102017122160.3

(51) Int. Cl. A61N 5/06; A61B 5/00

(52) U.S. Cl. CPC A61N 5/0603; A61B 5/0066; A61B 5/0084; A61N 2005/067; A61N 2005/0611; A61N 2005/063; A61N 5/062

(57) ABSTRACT

The present invention relates to an endoscope (2) having first conduction means configured to transmit electromagnetic waves between an illumination device (40) for illuminating an observation area (41) and a distal end (5) of the endoscope (2), and second conduction means configured to transmit electromagnetic waves between a therapy device (42) for treating a therapy area (43) within the observation area (41) and the distal end (5) of the endoscope (82). The endoscope is characterized by a third conduction means configured to transmit electromagnetic waves between an optical coherence tomography device (37) by means of which depth information about said area can be obtained during treatment of the therapy area (43) and the distal end (5) of the endoscope (2).

### TECHNICAL FIELD

The present invention refers to an endoscope.

### BACKGROUND

Endoscopes are devices with which the interior of living organisms or also technical cavities can be examined or even manipulated. Rigid and flexible endoscopes are known.

The different types of endoscopes are adapted to the special conditions of the organ in which they are used due to their diameter, length, flexibility or other properties.

An example of an endoscope in which a diagnostic function as well as a therapy function is implemented is for instance known from the German utility model application 9112188.4. The endoscope known from this document forms the preamble of claim 1.

### SUMMARY

This document describes a system with a therapy laser device, a diagnostic laser device and a cold light source. The diagnostic laser device as well as the therapy laser device are designed as separate solid-state lasers. The corresponding therapy laser, the diagnostic laser or the cold light are coupled into the endoscope via light wave guides. At its distal end, the endoscope has a tubular design in the manner of a tube. The tube can be inserted into the corresponding cavity of the patient. The distal end of the tube is placed near a location where a tumor diagnosis is to be performed and where the respective tumor is to be treated later. A control unit can be used to control a diagnostic mode, with which the tumor is made visible, and a therapy mode, in which the tumor is irradiated with high-energy radiation and thus denatured, for example. The visualization of the site is done via an image processing program and can be shown on a display. For therapy, the so-called Photodynamic Therapy (PDT) is used with this system and for diagnostics, the so-called Photodynamic Diagnostics (PDD) is used with this system. A photosensitizer is applied to the place to be treated or observed with which the laser radiation interacts. Based on this system it is the object of the present invention to provide an endoscope with which the visualization of the area to be treated is improved, especially during therapy. To solve the problem, this invention proposes an endoscope with the features of claim 1.

The endoscope has a first conduction means, a second conduction means and a third conduction means. By means of the first conduction means, electromagnetic waves or electromagnetic beams can be transmitted between an illumination device and a distal end of the endoscope. By means of the illumination device, an observation area is directed to the location that illuminates the distal end of the endoscope. An illumination can be on the one hand an illumination of the examined area and/or an illumination to increase the contrast, which is provided in addition to an illumination.

As an endoscope, for example, an assembly of an endoscope body which, for example, forms a tube in which, for example, already known functionalities are integrally integrated and the insert or housing described later, can be understood.

An endoscope, for example, is a device with which the interior of living organisms, but also technical cavities, can be examined or even manipulated. Any device with which this is possible can be seen as an endoscope.

In medicine, endoscopes are used to examine and manipulate or treat various internal areas of the human body. For example, so-called bronchoscopes are used for the diagnosis and therapy of tumors in the trachea and within the bronchial tree. A so-called gastroscope is usually used for the diagnosis and therapy of tumors of the mucous membrane of the oesophagus, stomach and duodenum. Rectoscopes can be used to diagnose and treat tumors of the rectum. Cystoscopes can be used for the diagnosis and therapy of tumors of the urinary bladder interior.

The different types of endoscopes mentioned above are adapted to the specific conditions of the organ for which they are used due to their diameter, length, flexibility or other properties.

Illumination or lighting equipment can be understood to mean, for example, any device that illuminates the sample in any way. This means e.g. a lighting means for illumination, which e.g. applies radiation e.g. light to a larger area than the field of view determined by an optical system of the endoscope or a CCD-chip.

Illumination respectively lighting equipment can also be understood to mean, for example, lighting for increasing contrast, which, for example, acts on an area within the field of vision and increases the contrast in this area. The lighting for illumination can be combined with lighting to increase contrast.

A lighting device illuminates a section of the specimen in general. The lighting device can have a lighting source that illuminates an area on the sample in the manner of a spotlight.

Several spotlights can be provided on the sample, which, for example, also interact overlapping on the sample. For example, a smaller spotlight can be provided within a larger spotlight. Each of the spotlights can come from different corresponding light sources or from one corresponding light source.

A spotlight can be generated e.g. by visible light e.g. white light. A spotlight can also be generated by light of certain wavelengths, e.g. light for Narrow Band Imaging (NBI) and/or light for the excitation of a photosensitizer for Photodynamic Diagnostics (PDD). For example, the larger of at least two spotlights can be generated by white light and/or NBI light and/or PDD light and the smaller of at least two spotlights can be generated by white light and/or NBI light and/or PDD light.

The larger of the at least two spotlights illuminates the area observed by the optics, and the smaller spotlight can be fully or partially provided within the larger spotlight, especially fully or partially within the field of view.

The larger spotlight is defined e.g. as illumination for floodlightning and the smaller spotlight is defined e.g. as illumination for contrast enhancement. This is because the overlay of the two spotlights always leads to an increase in contrast in the superimposed area. It is advantageous if spotlights with different properties (e.g. wavelengths such as NBI light, PDD light, white light) are superimposed in the superimposed area.

The above-mentioned illuminations can be guided through the first conduction means.

If both types of lighting are provided, a further e.g. fourth conduction means can also be provided in which the other type of lighting is guided.

These conduction means are provided at least in the area of the endoscope or run through it. If, for example, some or more or all of the aforementioned conduction means are provided within the insert inserted into the endoscope described in detail later, one or more further conduction means may be provided in the endoscope body which, for example, contains a channel or working channel into which the insert or the housing described later below is inserted.

Within this observation area a therapy area can be delimited from a therapy beam, e.g. according to the type of point or spotlight. In this therapy area, the tissue is treated e.g. by energetic radiation, in particular manipulated and/or destructively treated. The therapy beam, for example, is generated in a therapy device that can be coupled with the endoscope. In the therapy device, electromagnetic waves or electromagnetic beams with a predetermined wavelength and/or in predetermined wavelength ranges are generated and transmitted via the second conduction means from the therapy device to the distal end of the endoscope. In addition to this therapy beam and the illumination beam, which are transmitted by the first conduction means and by the second conduction means respectively, electromagnetic waves and electromagnetic beams, respectively, which are generated in an optical coherence tomography device, are transmitted by means of a third conduction means to the distal end of the endoscope and of the insert provided in the endoscope, respectively. By means of optical coherence tomography, depth information, for example on tumor depth, can be obtained at least in the therapy area. The depth information obtained by optical coherence tomography can, for example, be visualized on a screen after image processing.

This invention combines the functionality of optical coherence tomography with a therapy and illumination function of the endoscope for the first time, and during therapy depth information about the therapy area can be obtained and, for example, visualized. This makes it easier to decide during therapy whether the therapy is effective and how the therapy beam can be guided for therapy.

Thus, in addition to the two-dimensional visualization during the therapy, a third piece of information, namely the depth information in the area of the therapy, can be obtained immediately, which can contribute to an improved tumor treatment.

Observation by means of optical coherence tomography during therapy can also include procedural guidance in which, at intervals of a few seconds, milliseconds or nanoseconds, e.g. 1 to 5, in particular 1 to 2 seconds, milliseconds or nanoseconds, it is switched between the operation of the therapy beam and the operation of the optical coherence tomography, whereby, however, for the user, for example on a screen implemented in the system, the optical impression is created that the image of the optical coherence tomography is taken during the therapy. In addition to this sequential procedure, a simultaneous procedure can also be applied, whereby the optical coherence tomography is operated directly simultaneously with the operation of the therapy device.

As far as the first, second and third conduction means are concerned, more than these three conduction means can also be provided. These conduction means may each be designed as separate elements or may be provided integrally with each other in any combination. The definition of the conduction means is a functional definition. If an element fulfills the function of the first and/or second and/or third conduction means, it is to be seen as an integral conduction means.

The therapy range can lie within the observation area, then the third conduction means is configured to transmit electromagnetic waves between an optical coherence tomography device and the distal end of the endoscope, by means of which depth information about this area and the observation area or at least parts of the observation area can be obtained during the treatment of the therapy area. If the therapy area lies within the observation area, which is advantageously the case, then parts of the observation area are also imaged using an optical coherence tomography device.

It may be advantageous if at least two, preferably three, of the following distal ends are integrally provided in a tube of the endoscope: a distal end of the first conduction means, a distal end of the second conduction means, a distal end of the third conduction means. Such a tube can be used e.g. as a housing of the endoscope, this tube can have e.g. one or more channels, e.g. working channels. In such a channel, e.g. the housing or insert mentioned below can be used, which contains at least two of the conduction means.

It can also be advantageous if at least two of the following elements are provided within, in particular integrally in a common housing: a distal end of the first conduction means, a distal end of the second conduction means, a distal end of the third conduction means. In particular, the housing may have such an outer diameter that it can be inserted into a working channel of the endoscope, in particular of an endoscope designed as a cystoscope. In a cystoscope, the working channel diameter is very small so that this cystoscope can be inserted through the narrow urethra at all.

It is possible that a respective proximal end of the conduction means protrudes out of this housing, e.g. at the rear, and is combined, for example, with a proximal end of another of the conduction means, e.g. in a hose. If at least two conduction means are combined in a common housing, they can easily be attached to the endoscope together.

The present invention, for example, is the first invention to provide a housing as an embodiment of the invention in which at least two functionalities, e.g. therapy, observation and optical coherence tomography, are combined. It may be particularly advantageous if at least one of the at least two conduction means provided in the housing is the conduction means for transmitting the electromagnetic waves from the optical coherence tomography device. It can also be advantageous if all three conduction means with their distal ends are provided in this housing. This requires further miniaturization of the corresponding elements.

As a further possible embodiment, the first and second conduction means can be combined in a multi-clad, in particular double-clad fiber.

A so-called double-clad fiber (DCF) can be a fiber, in particular glass fiber, which is composed of a core glass or core material (fiber core) and at least two cladding glasses or cladding materials (first fiber cladding and second fiber cladding), the inner cladding glass (first fiber cladding), which encloses the core glass, and the outer cladding glass (second fiber cladding), which encloses the inner cladding glass.

For example, all three glasses have different refractive indices. The core glass corresponds e.g. to that of the single mode fiber. For example, the inner cladding glass has a lower refractive index than the outer cladding glass.

The electromagnetic wave for lighting or the illumination beam can have a homogeneous angular distribution for the purpose of homogeneous lighting/illumination and can be guided in the fiber core and/or in the inner cladding glass and can be reflected by the outer fiber cladding, while the therapy beam or the electromagnetic waves for therapy, for example, is guided exclusively in the core, i.e. reflected by the first fiber cladding. As a synonym for Multi-clad Fiber, the term “Mehrmantelfaser” is also used, or as a synonym for the term Dual-clad fiber, the term “Zweimantelfaser” fiber is also used. As far as the term Multi-clad fiber is used, more than two light wave or electromagnetic wave conducting claddings are provided.

By integrating the first and second conduction means in a dual-clad or multi-clad fiber or also in a common fiber bundle, further miniaturization can be achieved and it is also ensured that the therapy area is centrally located in the lighting area. Preferred acceptance angles of the fiber core for the therapy beam are 3-9°, 4-8° and 5-6°. Preferred acceptance angles of the cladding core are 20-60°, 30-50° or 40°. The acceptance angle is the maximum angle of incidence with which light can be fed into the glass of an optical fiber and guided there.

A further possibility of cost savings and miniaturization can be the coupling into a single-clad fiber with a very high acceptance angle in the form that the electromagnetic radiation of the illumination source is coupled strongly divergently with a homogeneous angular distribution, but the therapy source is coupled slightly divergently into the same single-clad fiber. The preferred half acceptance angle is 20-60°, 30-50° or preferably 40°.

A favorable configuration of the third conduction means at its distal end inside the housing may be as follows. The third conducting means may comprise a movable mirror and a deflecting device within this housing. These elements are configured such that the electromagnetic waves of the optical coherence tomography device, i.e. the optical coherence tomography beam, are transmitted via the deflector to the movable mirror and reflected therefrom in such a way that it exits through a first opening in a front surface of the housing. The optical coherence tomography beam can emerge from the opening in such a way that it can be irradiated into the observation area. A very compact design can be achieved by the construction of the movable mirror in connection with the deflecting device. In addition, the movable mirror may reciprocally move the optical coherence tomography beam in a scanning motion on the surface of the area examined by the endoscope to receive information for an optical coherence tomography image. Preferably the movable mirror is connected to the control means which controls a movement of the mirror. The surface is the surface of the area examined by the endoscope. For example, the mirror is part of a so-called MEMS system (micro-electromechanical system), where the mirror can be moved by electromagnetic forces. The mirror and the control electronics can be part of the MEMS system, which is provided in the insert or housing. The housing can therefore serve as a kind of support for the MEMS system. Possibly, the movable mirror can be configured in such a way that an at least one-dimensional scan can be carried out and the OCT beam (optical coherence tomography beam) follows a line in the observation area at least in one scan. The line can lead through the therapy area. In this way, a two-dimensional depth profile can be obtained along the therapy area and the observation area. The observation area can, for example, include a tumor completely or partially.

The term “at least one-dimensional” means that a two or multi-dimensional screening or a two or multi-dimensional scan are not excluded. A two-dimensional scan provides a three-dimensional image with respect to the area and depth of the observation area and can lead to an even more detailed determination of the tumor extent and the therapy progress or end point in the observation area.

In particular, the movable mirror can be configured in such a way that it performs an at least one-dimensional scan through the housing at a distance of 5-20 mm from the distal end of the housing during the treatment of the therapy area, which scan completely encloses the therapy area and the observation area in one direction.

The therapy beam formed by the electromagnetic waves for the treatment of a therapy area can intersect at a distance of between 5 and 15 mm from an extension end of the first and second line device with the illumination beam formed by the electromagnetic waves for illuminating an observation area.

As another possible configuration of the third conduction means, this conduction means may comprise a fiber from whose distal end the optical coherence tomography beam is directed onto the deflecting device so that the beam is deflected via the deflecting device onto the movable mirror. A so-called grin lens (gradient lens) can be provided between the distal end of the fiber and the mirror. In such a lens, the optical properties of continuous material transitions are used. Grin lenses are, for example, cylindrical, transparent optical components with a refractive index decreasing in the radial direction. Usually the refractive index decreases quadratically with the distance to the center (parabolic function). A short rod made of such a material acts like a common converging lens, but has flat surfaces on the light entry and exit sides. This facilitates assembly, miniaturization and connection with subsequent optical elements. The flat surface of such lenses is an advantage over conventional lenses, especially when coupled to optical fibers.

The distal end of the fiber from which the OCT beam is decoupled can be located eccentrically inside the housing. This design allows sufficient space for the movable mirror to remain in the center axis of the housing so that it can be used in a scanning configuration. The combination of the eccentric fiber with the deflector and the mirror results in a very compact design, and the beam emerges, for example, from the front surface of the housing in a plane that encompasses the central axis of the housing.

It has been found that it can be beneficial to place the movable mirror at an angle to the central axis of the housing. This makes it possible to redirect the optical coherence tomography beam so that it emerges from the opening in the front surface of the housing at a predetermined angle to the central axis of the housing and/or to an axis of the therapy and/or observation beam. If the therapy beam and the optical coherence tomography beam run at just this adjustable angle to each other, a situation can be created in which the optical coherence tomography beam crosses the therapy area on the tissue surface during the at least one-dimensional scan along a line. If the corresponding conduction means are provided in the same endoscope, this opens up the possibility of generating an optical coherence tomography image of the treated area while the therapy beam is used for therapy.

Furthermore, it can be advantageous that the MultiClad fiber, in particular the Double-clad fiber, in which the second and first conduction means are combined, is also provided eccentrically within the housing. With this design, the decoupled electromagnetic waves or the therapy beam and the illumination beam can be decoupled parallel to the central axis of the housing and delimit the therapy area or observation area. The eccentric arrangement of the fiber creates space inside the housing to implement the functionality of the optical coherence tomography in the housing.

Furthermore, it can be advantageous that the MultiClad fiber, in particular the Double-clad fiber, in which the second and first conduction means are combined, is also provided eccentrically within the housing. With this design, the decoupled electromagnetic waves or the therapy beam and the illumination beam can be decoupled parallel to the central axis of the housing and delimit the therapy area or observation area. The eccentric arrangement of the fiber creates space inside the housing to implement the functionality of the optical coherence tomography in the housing.

It can be advantageous if the end of the MultiClad fiber, especially the Double-clad fiber, projects beyond the front surface of the housing. This prevents the housing from influencing the therapy or illumination beam. The front of the housing itself can assume a lens function for the OCT beam so that it is focused on the tissue.

It has also turned out to be possible that the therapy area lies centrally within the observation area. If the illumination beam is configured in such a way that it covers a larger area than the therapy beam, it is advantageous for good observation of the area to be treated if the therapy beam is relatively central, i.e. in the middle, in the illuminated observation area. It has turned out that the housing can have a diameter in particular in the range of ≤2 mm. As far as the housing diameter is used, this means the longest distance in cross-sectional direction to its longitudinal axis. The housing may be cylindrical in cross-sectional direction, in particular round or oval in shape.

Any lighting system may be used for the lighting device. In particular, it is advantageous to use a narrow band imaging (NBI) system, a red-green-blue (RGB) system, a photodynamic diagnostic (PDD) system and/or a white light system. These lighting devices can each be used as a means to illuminate the sample in any way.

It is also possible to provide several spotlights on the sample which, for example, also interact overlappingly on the sample. For example, a smaller spotlight can be provided within a larger spotlight. Each of the spotlights can come from different corresponding light sources or one corresponding light source.

Each spotlight can be generated by one of the aforementioned systems or one of the aforementioned systems can also generate at least two spotlights.

An RGB system uses a so-called RGB laser, which has a red, green and blue laser module. Most color gradations can be mixed by modulating the wavelengths from the individual laser modules. With a typical analog modulated RGB laser, which has 256 brightness levels for each color, 16,777,216 different colors can be displayed. In this way, the individual colors can be adapted to the tissue to be treated and the contrast in the observation area can be increased. The RGB system can also be adjusted to fulfill the NBI functionality or the PDD functionality, e.g. by mixing the laser components.

A cold light lamp or any other means for producing white light can be used as a white light system. The white light can also be generated by the RGB system.

The RGB system can have a red laser operating in the wavelength range from 600 nm to 699 nm, a green laser operating in the wavelength range from 500 nm to 599 nm, and a blue laser operating in the wavelength range from 400 nm to 499 nm.

In a so-called PDD system, a photosensitizer or a precursor of a photosensitizer is applied to the corresponding area, which, for example, actively accumulates in or on the tumor cells. The sensitizer molecules are made to fluoresce, for example, by irradiation with light of a certain wavelength. This fluorescent light can achieve a better contrast. A PDD system can therefore be a system that has a radiation source with a specific radiation adapted to a photosensitizer. The photosensitizer to be applied may be part of the PDD system. The PDD light can also be generated by the RGB system.

A so-called NBI system is a system that is adapted to emit beams of narrowband light from e.g. two wavelengths, especially blue light of e.g. 415 nm and green light of e.g. 540 nm. NBI light from these two wavelengths is absorbed by vessels, for example, but reflected by the mucous membrane. This is a great advantage to increase the maximum contrast in the observation area. In an NBI system at least one radiation source is provided which emits radiation of at least two different wavelengths which are coupled in via the first conductivity device. The NBI light can also be generated by the RGB system.

If the PDD system or NBI system is used, the RGB system can be configured to perform these functions.

With the PDD system and the NBI system described, as well as mixing specific colors with the RGB system, the contrast in the observation area can be increased. These systems are therefore a lighting device with an illumination to increase the contrast. This can, for example, be provided in addition to lighting for illumination. A lighting device for illumination is referred to in this context as a second lighting device, and a lighting device for increasing contrast is referred to in this context as a first lighting device.

The light radiation reflected by the tissue can be transmitted to an image processing device via the first conduction means and/or any other of the existing conduction means. Alternatively or additionally, a sensor, e.g. an objective and/or CCD sensor, can be provided at the distal end of the endoscope, which picks up the radiation and transfers it to the image processing device.

In contrast to the illumination beam, the therapy laser, for example, has a different wavelength and/or different intensity, so that the tumor or the material to be treated can be treated with it, in particular resected, vaporized or coagulated. Preferred wavelengths of the therapy beam are 400 nm, 450 nm, 532 nm, 780 nm, 1064 nm, 1470 nm, 1900 nm, 2200 nm, 3000 nm, 10,600 nm. These values can form an upper or lower limit of a preferred range. Preferred intensities of the therapy beam are: 1 W, 5 W, 10 W, 20 W, 50 W, 100 W and 200 W. These values can each form an upper or lower limit of a preferred range.

The therapy beam can be decoupled from the therapy device with an opening angle of less than 9°. The aforementioned angle is the half angle (relative to the central axis). As a full angle, this would correspond to less than 18°.

Such a small angle ensures, for example, that the therapy spot is narrow in relation to the illumination spot.

A laser beam with an opening angle of more than 20° can be decoupled from the RGB system. The aforementioned angle is the half angle (relative to the center axis). This opening angle is larger than the opening angle at which the therapy beam is decoupled from the therapy device.

The therapy beam decoupled from the RGB system and the therapy beam decoupled from the therapy device can be coupled into the previously described single-clad fiber, dual-clad fiber or multi-clad fiber in such a way that the beam of therapy beam and illumination beam decoupled from the corresponding fiber has a homogeneous beam profile at a distance of between 5 and 15 mm from the decoupling point. Other preferred distances are 7.5 mm, 10 mm, 12.5 mm. These values can form upper or lower limits of a favorable range.

The single-clad fiber, the dual-clad fiber, or the multi-clad fiber, which form the first and second conduction means, can have a larger acceptance angle than the opening angle of the RGB system and/or the therapy equipment.

The acceptance angle is the maximum angle at which light can be coupled into the fiber. The acceptance angle is given as the half-angle relative to the central axis of the light conductor. The opening angle is the angle at which light exits a light source or fiber. The single-clad fiber or the dual-clad fiber, or the multi-clad fiber, which form the first and second conduction means, can be between 0.5 m and 10 m long. Other preferred lengths are 2 m, 4 m, 8 m. These values can form upper or lower limits of a favorable range.

A beam with an opening angle of more than 20° can be decoupled from the first and/or second conduction means. The aforementioned angle is the half angle (related to the central axis).

According to a secondary aspect of the invention, it indicates the entire system consisting of the endoscope, the illumination device, the therapy device and the optical coherence tomography device described above. In addition, a control device is provided in the system by means of which at least the illumination device, the therapy device and the optical coherence tomography device can be controlled.

Corresponding software modules may be provided in the control device or in addition to the control device for carrying out image processing for optical coherence tomography and/or image processing for an image of the observation area. The control device, the illumination device, the therapy device and the optical coherence tomography device may be provided in a uniform housing and may be located, for example, in a medical practice or clinic.

For example, one or more screens can be connected to this system to visualize the data.

A standard endoscope or cystoscope can, for example, be connected to the corresponding components of the system via a plug connection.

Thus, a modular structure has been achieved in which commercially available components can be combined with the new combination of optical coherence tomography therapy and illumination.

The problem mentioned above is further solved by an insert that can be fitted into the working channel of an endoscope. The insert has a housing with the first, second and third conduction means inside the housing. The first, second and third conduction means correspond to the conduction means previously described for the endoscope. Consequently, the corresponding features in the subclaims of the endoscope can also be combined with the corresponding features of the insert.

This insert is supplied, for example, as a separate part and can, for example, have one or more plugs at its proximal end by means of which the corresponding conduction means can be connected to the illumination device or the therapy device or the optical coherence tomography device. The illumination device, the therapy device, the optical coherence tomography device can be combined in one module with a common housing, which is provided in addition to an already existing module, which controls the functionalities already present in the tube of the endoscope. The module can be connected to the existing module in the doctor's office and can be operated, for example, via a PC or software provided in the existing module. This software is supplied, for example, with the insert and the module and can be installed on the PC in the practice or the existing module.

This makes it easy to combine existing endoscopes in the doctor's office or hospital with the functionality of optical coherence tomography and simultaneous therapy and illumination.

Alternatively, only the functionality of optical coherence tomography can be provided in one module in addition to the other module. It is also possible that the described functionality of optical coherence tomography is already present in the room in which the endoscope is used, and that the insert has a suitable connector system so that the conduction means can be connected to the corresponding equipment already present in the room or practice.

Thus the invention also covers a corresponding method of connecting an insect to already existing equipment.

### BRIEF DESCRIPTION OF THE DRAWINGS

Further advantageous embodiments and further developments of the invention result from the following embodiments in connection with the drawing:

FIG. 1 is a schematic illustration of a system for simultaneous observation and treatment,

FIG. 2a is a detailed view of a distal end of a cystoscope, wherein the insert according to the invention, which is also schematically shown in the Figure (but without its protective cover), is inserted into a working channel of the cystoscope,

FIG. 2b is a schematic view of the overlay of an illuminated field of view of the cystoscope, the illumination spot to increase contrast and the therapy laser spot,

FIG. 3a is a schematic drawing of an insert according to the invention, where the protective cover is shown here in contrast to the illustration from FIG. 2a,

FIG. 3b is an OCT image of a one-dimensional scan along the line Smax shown in FIG. 3a,

FIG. 4 is an overlay of the observation area of the therapy area with a one-dimensional scan line of the optical coherence tomography beam, and

FIGS. 5a to 5d show different combinations of therapy beam, its surrounding illumination beam to increase the contrast and the illuminated field of view.

### DETAILED DESCRIPTION OF EXAMPLE EMBODIMENTS

Before the system, the endoscope or the insert according to the invention are explained in more detail in the following using the examples shown in the Figures, the technology of so-called “Narrow Band Imaging” (NBI), the technology of photodynamic diagnostics (PDD), the use of an RGB system or a white light source and the technology of optical coherence tomography (OCT) are explained in more detail.

### Optical Coherence Tomography (OCT)

Optical coherence tomography is an examination procedure in which light of small coherence length is used with the aid of an interferometer for distance measurement in scattering material. The strengths of this method lie in the relatively high penetration depth of 1 to 3 mm into scattering tissue with a simultaneous high axial resolution of 0.5 to 15 μm and high measuring speed of 20 to 300 kvoxel/s. Compared to other methods such as MRT, it is also much cheaper and can be built much more compact. With optical coherence tomography, sectional and volume images of semi-transparent objects can be taken. The measuring principle is roughly comparable with ultrasound imaging. Both methods have the same reflection geometry. In optical coherence tomography, the structure in the sample is imaged by a transit time measurement similar to ultrasound imaging. The differences result from the high speed of light, since there are no electrical detectors that can measure transit time at such short distances. Therefore, the transit time is determined interferometrically as the transit time between the sample signals with a reference numeral. In order to achieve a high depth resolution and to filter out the background of strongly controlled light, light sources with short coherence lengths are used, in particular the so-called Time Domain OCT (TD-OCT) and the Frequency Domain OCT (FD-OCT) are distinguished today in optical coherence tomography.

In this case, a so-called optical coherence tomography beam is generated in the optical co-herence tomography device and, if necessary, irradiated via various optical elements onto the tissue to be examined.

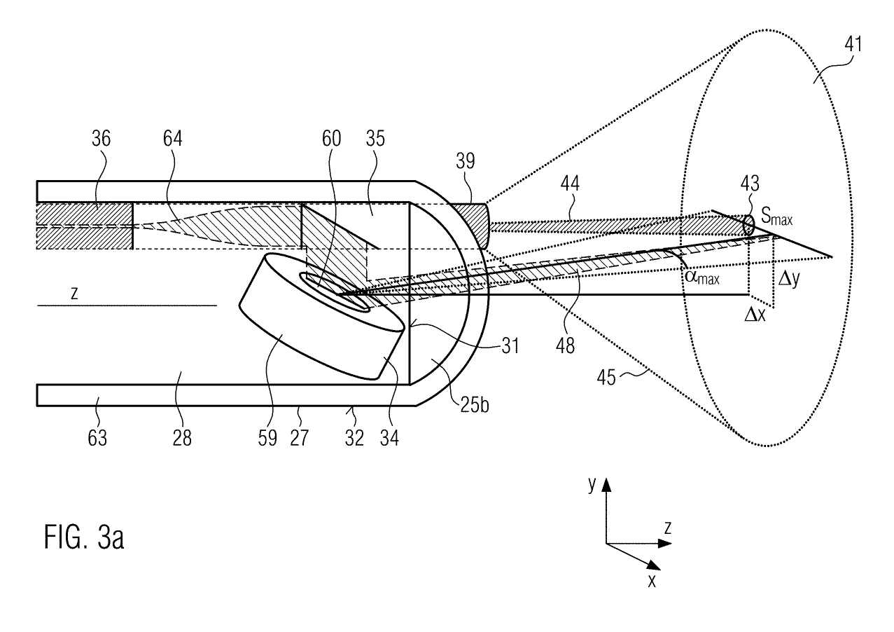

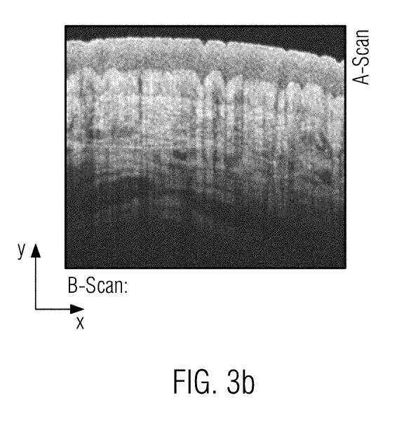

From the reflected radiation and by means of an interferometer, which can be contained in the optical coherence tomography device, a depth image can be obtained, as shown in FIG. 3b, for example. By scanning the optical coherence tomography beam along a line (FIG. 3b, for example, shows a scan along a line from 6 to 10 mm), a depth profile of the tissue in the area of the line can be obtained. By means of a two-dimensional resolution of the optical coherence tomography beam, such a three-dimensional OCT image can also be generated, unlike shown in FIG. 3b. Using appropriate image processing methods, the information extracted from the reflected OCT signal can be visualized on a screen and a two-dimensional depth profile or even a three-dimensional image of the area can be displayed, taking the depth into account.

The method of optical coherence tomography is combined with at least one therapy functionality and at least one illumination functionality.

### Narrow Band Imaging (NBI)

Narrow band imaging is an imaging technique for endoscopic diagnosis in which light of specific wavelengths, e.g. blue and green, is used to improve the surface contrast of tissue. For example, light with wavelengths of 415 nm (blue) and 540 nm (green) is applied to the surface to be examined. Since the strongest light absorption of hemoglobin occurs at these wavelengths, blood vessels are darkened and made more visible. Since tumor tissue usually differs from other tissue by stronger vascularization, the contrast between tumor and non-tumor tissue can be increased by irradiating the two different wavelengths (e.g. blue and green).

The NBI system can be adapted to emit beams of narrow-band light from, for example, two wavelengths, in particular, blue light of, for example, 415 nm and green light of, for example, 540 nm. In an NBI system, at least one radiation source is provided which emits at least radiation of two different wavelengths which are coupled in via the first conduction means. However, two radiation sources can also be provided for the different radiations.

The Narrow Band Imaging can be used to illuminate the observation area. This technique provides a lighting device with illumination to increase the contrast. This can, for example, be provided in addition to lighting for illumination. In this context, a lighting device for illumination is referred to as a second lighting device and a lighting device for increasing contrast is referred to as a first lighting device.

An image processing program can then be used to generate a corresponding image of the observation area from the reflection information of the illuminated area.

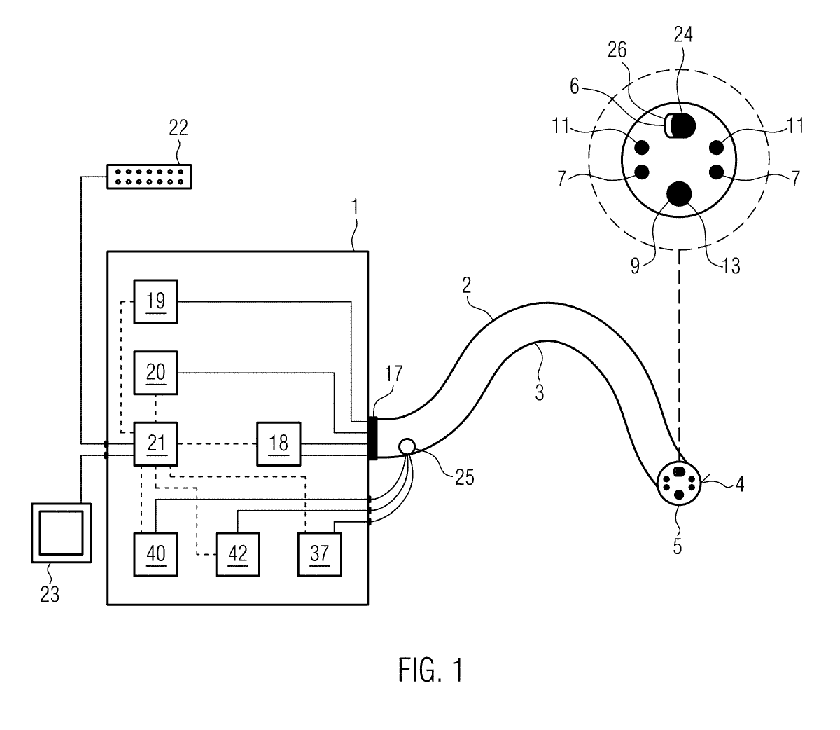

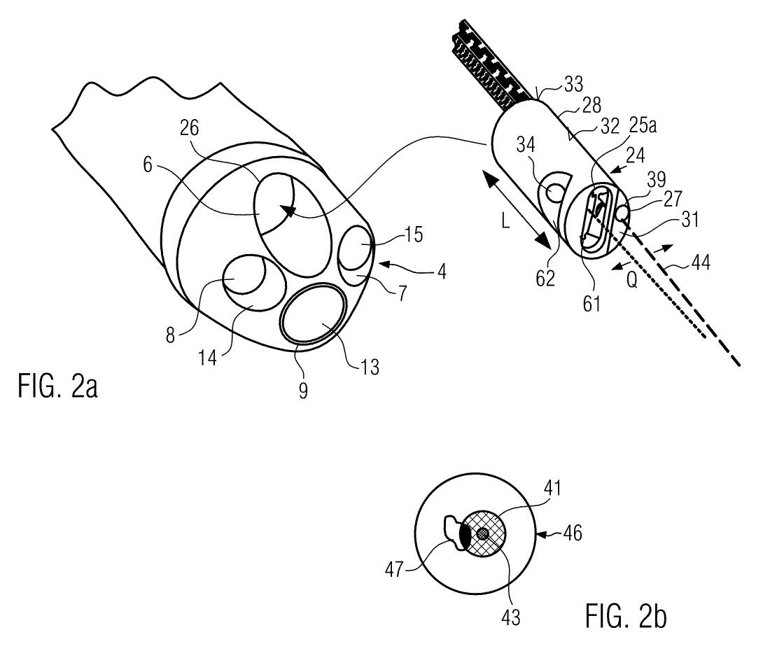

The illumination radiation reflected by the tissue can be transmitted to an image processing device via the first conduction means and/or any other of the existing conduction means. Alternatively or additionally, e.g. at the distal end of the endoscope, a sensor, e.g. a lens (see FIG. 1 and FIG. 2a: reference numeral 13) and/or CCD sensor can be provided, which picks up the radiation and transfers it to the image processing device.

### PDD (Photodynamic Diagnostics)

In photodynamic diagnostics, the contrast of tissue surfaces or the contrast between tumor-containing and non-tumor-containing tissue can also be improved. Here, a so-called photosensitizer or a precursor of a photosensitizer is applied to the corresponding area, which accumulates selectively in the surface of tumor cells. Irradiation with light of a certain wavelength causes the sensitization molecules to fluoresce. The contrast can be further increased by this fluorescence.

For the illumination in the endoscope according to the invention, such a wavelength can be used which is sensitive to a selected photosensitizer. This photosensitizer can, for example, be applied through another working channel of the endoscope, or applied through the working channel before the insert according to the invention is inserted into it.

In addition to this first illumination device to increase the contrast, a second illumination device can also be provided for illumination.

### RGB or White Light System

A so-called RGB system can also be used to illuminate or improve the contrast of the observation area.

An RGB system uses a so-called RGB laser, which has a red, green and blue laser module. Most color gradations can be mixed by modulating the wavelengths from the individual laser modules. With a typical analog modulated RGB laser, which has 256 brightness levels for each color, 16,777,216 different colors can be displayed. In this way, the individual colors can be adapted to the tissue to be treated and the contrast in the observation area can be increased.

This RGB laser can also be used, for example, for the NBI method as well as the PDD method, by completely switching off one or two of the laser modules or by mixing the radiation from the corresponding laser modules accordingly.

### Endoscope

An endoscope is a device with which the interior of living organisms as well as technical cavities can be examined or even manipulated. Any device with which this is possible can be seen as an endoscope.

In medicine, endoscopes are used to examine and manipulate or treat various internal areas of the human body. For example, so-called bronchoscopes are used for the diagnosis and therapy of tumors in the trachea and within the bronchial tree. A so-called gastroscope is usually used for the diagnosis and therapy of tumors of the mucous membrane of the oesophagus, stomach and duodenum. Rectoscopes can be used to diagnose tumors of the rectum. Cystoscopes can be used for the diagnosis and therapy of tumors of the urinary bladder interior.

The different types of endoscopes are adapted due to their diameter, length, flexibility or other properties to the special conditions of the organ in which they are used.

The endoscope may have at least one tube 2 described below. An insert 24 described below can be inserted into a working channel 6 of the tube. Thus, for example, elements inserted in the working channel or in other channels running through the tube are also seen as part of the endoscope.

A first, second and third conduction means can at least be assigned to the tube, e.g. run through it. If such a conduction means runs through the tube, it may be integrally and/or loss-proof connected to the tube, or it may be loose, e.g. movable in the longitudinal direction in the channel, such as the working channel

As far as the first, second and third conduction means are used, more than these three conduction means can also be provided. These conduction means may be designed as separate elements or may be provided integrally with each other in any combination. The definition of the conduction means is a functional definition. If an element fulfills the function of the first and/or second and/or third conduction means it is to be seen as an integral conduction means.

### Illumination/Increased Contrast/Field of View Illumination

Illumination can be understood as any means of illuminating the sample in any way.

This means e.g. an illumination for floodlightning an area, which e.g. applies radiation e.g. light to a larger area than the field of view determined by the optics or the CCD chip.

This can also include, for example, illumination to increase contrast, which, for example, affects an area within the field of vision and increases the contrast in this area.

The illumination for floodlightning an area can be combined with the illumination for contrast enhancement.

The aforementioned illumination can be routed through the first conduction means. If both types of illumination are provided, a further, e.g. fourth conduction means can also be provided in which the other type of illumination is guided.

### Relevance for the Visualization and Therapy of Bladder Carcinomas

In particular, the system according to the invention is used in conjunction with a cystoscope for the examination and treatment of bladder carcinomas. Every year, approximately 0.5 million people worldwide are newly diagnosed with bladder cancer. 70-75% of tumors are not muscle-invasive and can be treated by transurethral resection. In 15-61% of cases, however, relapses occur within one year, in 31-78% within 5 years, making bladder cancer the most expensive type of cancer. As a precaution, a cystoscopy with a flexible cystoscope without anesthesia is carried out by established urologists at 3-12 month intervals. If a relapse is detected, it would be switched to a rigid endoscope for treatment with electrosurgical devices, which is why the patient and urologist usually opt for in-patient treatment under anaesthesia, which is considerably more expensive for the health care system.

The embodiment according to the invention can, for example, significantly increase the visualization of relapse during treatment and the acceptance of direct out-patient treatment compared to renewed in-patient treatment and reduce the relapse rate.

The laser light that can be used for the therapy beam is extremely efficient and it is possible to guide it to the treatment site via the light guide of a flexible cystoscope. A simultaneous visualization of the tumor and the tissue interaction for monitoring the treatment endpoint using optical methods can make the application more precise and gentle for the patient.

For use with a flexible cystoscope, an insert designed in the manner of a thin catheter can be provided for this purpose.

First embodiment of an observation and treatment system according to invention

Schematically, a first example of an inventive observation and treatment system is shown in FIG. 1.

This shows a base station with reference numeral 1, to which an endoscope 2 is connected. Endoscope 2 has a tube 3. In the present case, the endoscope is a flexible endoscope, which is to be indicated by the serpentine shape of the tube 3. A rigid endoscope can also be used in this invention.

The tube 3 has six channels, whose exit openings are shown schematically in FIG. 1 on a front surface 4 at a distal end 5 of the endoscope 2. In the alternative embodiment from FIG. 2a, however, only the openings of four channels can be seen, since the fifth and sixth channels 11, 12 through the Bowden cables described below have no exit opening at the distal end of tube 3. Such Bowden cables are not necessarily provided for in the endoscope. A rigid endoscope, for example, does not have such Bowden cables. The Bowden cables are only an example for the mobility of the tube 3. They can be connected via a Bowden cable control unit 18 of the base station. If a rigid instrument is used, they would be omitted.

In the embodiments from FIGS. 1 and 2a, a first channel is formed as working channel 6, through which the insert 24 of the endoscope 2, explained below for example, can be passed.

In a second and third channel 7, 8 an optical fiber is guided, which can illuminate the visible area with the endoscope e.g. by means of white light.

A fourth channel 9 is formed as a camera channel In this case, a lens (see FIG. 1 and FIG. 2a: reference numeral 13) can be provided as sensor, which picks up the radiation and transfers it to an image processing device. At this point, a CCD chip or another sensor can also be placed in the opening to visualize the visible area of the cystoscope. A complete micro-camera can also be provided there.

In the fifth and sixth channels 11, 12 there are Bowden cables, so that the tube 3 can be adjusted in its shape by actuating the Bowden cables due to its flexibility. In FIG. 1, these Bowden cables are connected to a Bowden cable control unit 18 provided in the base station.

A detailed embodiment of a distal end 5 of the endoscope is shown in the magnification in FIG. 1 top right and a second embodiment of the distal end 5 of the endoscope is shown in FIG. 2a.

In contrast to the embodiment from FIG. 1, the front surface 4 of the embodiment in FIG. 2a is rounded towards the front with smaller bulges at the points where the second channel 7, third channel 8 and camera channel 9 end.

In FIG. 2a, the objective 13 and the illuminating fiber guided in the second and third channel 7, 8 respectively are firmly connected to the tube for illumination of the visible area and are delivered as an integral component.

These illumination fibers are intended for the transmission of an illumination. This illumination can be provided in addition to the illumination to increase the contrast, or also without a further lighting functionality provided in the insert or elsewhere.

Various means of manipulation can be used in working channel 6, e.g. the insert 24 described below.

As shown schematically in FIG. 1, the tube 3 can have a multi-pole plug 17, via which corresponding conduction means (e.g. the illumination fibers in the second or third channel 6, 7 and the cable to the objective 13) can be connected to the base unit 1, preferably to corresponding devices which interact with the illumination fibers or the objective.

In this case, the multi-pole plug 17 connects the Bowden cable control unit 18, a cold light lamp 19, which represents a second illumination device for illuminating the field of view, and a camera unit 20.

The corresponding connection for the transmission of the signals or electromagnetic waves or forces from/to the Bowden cable control unit 18, the cold light lamp 19 or the camera unit 20 to the endoscope 2 is shown schematically by the solid lines inside the base unit 1.

The insert 24 shown in FIG. 2a, for example, is supplied as a separate part and may, for example, have one or more plugs 24a, 24b, 24c at its proximal end, by means of which the corresponding conduction means (first, second, third conduction means) can be connected to the illumination device (in this case the illumination device for contrast enhancement 40) or the therapy device 42 or the optical coherence tomography device 37.

In this example, the illumination device for increasing contrast 40, the therapy device 42, the optical coherence tomography device 37 are provided within a common module with the cold light lamp 19, the camera unit 20, and the Bowden cable operating unit 18.

The illumination device for increasing contrast 40, the therapy device 42, the optical coherence tomography device 37 can also be combined in a module with a common housing, which is provided in addition to an already existing module, which controls the functionalities already present in the tube 3 of the endoscope 2. The module can be connected to the already existing module in the doctor's office and can be operated, for example, via a PC or software provided in the already existing module. This makes it easy to combine existing endoscopes in the doctor's office or hospital with the functionality of optical coherence tomography and simultaneous therapy and illumination.

Alternatively, only the functionality of optical coherence tomography can be provided in one module in addition to the other module. It is also possible that the described functionality of optical coherence tomography is already present in the room in which the endoscope 2 is used and that the insert 24 has a suitable plug system so that the conduction means can be connected to the corresponding equipment already present in the room or practice.

The Bowden cable control unit 18, the cold light lamp 19 and the camera unit 20 are connected via a common control unit 21. The functions of the camera unit 20, the cold light lamp 19 and the Bowden cable control unit 18 can be controlled centrally in the control unit. An input station 22, such as a keyboard and/or mouse, is connected to the control unit 21 for this purpose. In addition, control unit 21 can be connected to a visualization unit 23, which can be provided externally from base station 1. This may be a simple PC screen.

As an alternative to the control unit being provided in the base station 1, it can also be provided by a PC connected to the base station or the aforementioned modules.

The camera unit 20, cold light lamp 19 or Bowden cable control unit 18 shown in the Figure, the illumination device for contrast enhancement 40, the therapy device 42, the optical coherence tomography device 37 can also be combined in one module with a common housing, either in each case or in any combination with each other. In this case, it is advisable not to provide a multi-pole connector 17 at the proximal end of the tube, but to provide several connectors at the proximal end of the tube, which can be coupled with the respective modules. In this way, the system can be made up of several modules that can be easily replaced individually.

As shown in the examples in FIGS. 1 and 2a, the insert 24 is inserted in working channel 6. As schematically shown in FIG. 1, this insert 24 can be inserted into the working channel 6 through a lateral opening 25 in the proximal end of the working channel 6 and passed through the working channel 6 in such a way that it protrudes from the distal end 5 of the endoscope 2 at a front opening 26 of the working channel 6 provided in the front surface 4 of the tube 3.

The insert 24 is seen together with the tube 3 as an “endoscope”. The endoscope 2 in the present case thus contains the tube 3, in which at least the objective 13 of the camera unit 20 is integrally provided, and the two illumination fibers for illuminating the visible area.

The first, second and third conduction means according to the invention are shown in the embodiment, as schematically shown in FIGS. 2 and 3a, in the insert 24.

As shown in FIG. 2a, the insert 24 has a housing 28 at its distal end 27. The housing 28 has a cylindrical, in particular circular, elongated shape. However, other embodiments are conceivable for this housing. The housing may also have an oval, angular or any other shape in its cross-sectional direction transverse to the longitudinal direction.

It has turned out that the housing 28 can have a diameter of 2 mm in the range of <in particular. As far as it is geared to the housing diameter, this is the longest distance in cross-sectional direction to its longitudinal axis.

With the aforementioned diameters it is possible that the housing, which contains at least two of the first, second or third conduction means, can be inserted into existing working channels of cystoscopes available on the market. The housing can have a cylindrical shape in its cross-sectional direction, in particular a round or oval shape. The housing 28 can have an elongated design in its longitudinal direction. The housing 28 serves as a holder for the movable mirror 34. The mirror 34 is part of a so-called MEMS system (micro-electromechanical system), whereby the mirror 34 can be moved by electromagnetic forces. The mirror 34 and the control electronics are part of the MEMS system, which is provided in the housing. The housing can thus serve as a kind of mounting for the MEMS system. In addition, the housing 28 can also be used as a holder for the corresponding conduction means, available for the OCT beam, for the therapy beam, and for the light beam to increase contrast. The housing 28, which serves as a holder, can also be surrounded by a protective cover 63 (see FIG. 3a). This protective cover 63 is not shown in FIG. 2a. In FIG. 3a, on the other hand, it can be seen that the protective cover 63 accommodates the housing 28. The protective cover can have a tight, especially waterproof design. For this reason, the front end of the protective cover 63 is shown closed in FIG. 3a. The insert, for example, represents the unit consisting of housing 28, protective cover and cables protruding from housing 28 and protective cover 63. The longitudinal direction of the tube 3, the endoscope 2, the insert 24 and the housing 28 indicates the direction in which the corresponding conductors, such as the line fibers and/or cables, are routed through the endoscope 1.

In FIG. 2a, the longitudinal direction in the area of the insert 24 is marked with the reference numeral L. In contrast, the transverse direction Q denotes a direction perpendicular to the longitudinal direction L and transverse to the longitudinal direction (see FIG. 2a).

The housing 28 can be a metal housing or a plastic housing, or parts of it can be made of these described materials. A biocompatible material is preferred for the housing 28.

The housing 28 has a front surface 31, a peripheral surface 32 and a rear surface 33.

In the example in FIG. 2a, the front surface 31 and the rear surface 33 are planar. In particular, in the embodiment in FIG. 2a, the OCT beam emerges from a free opening 25a in the front surface 31 of the housing. As shown in FIG. 3a, a lens 25b is provided on the front surface 31 of the housing 28, through which the OCT beam is guided. The configuration of the insert or of the housing 28 provided at the front side of the insert or of the protective cover 63 is explained in more detail below using FIG. 3a.

This shows a cross-section through the distal end of the insert. This has the protective cover 63, which accommodates the housing 28. A movable mirror 34, a prism 35 and a fiber 36, hereinafter referred to as optical coherence tomography fiber, form a third conduction means for transmitting electromagnetic waves from the optical coherence tomography means 37 (cf. FIG. 1) to the distal end 5 of the endoscope 2. A so-called grin lens 64 (gradient lens) is provided between the distal end of the fiber 36 and the mirror 34, in particular between the distal end of the fiber 36 and the prism 35. In such a lens, the optical properties of continuous material transitions are used. Grin lenses are, for example, cylindrical, transparent optical components with a refractive index decreasing in radial direction. In most cases, the refractive index decreases quadratically with the distance to the center (parabolic function). A short rod made of such a material acts like a common converging lens, but has flat surfaces on the light entry and exit sides. This facilitates assembly, miniaturization and connection with subsequent optical elements. The flat surface of such lenses is an advantage over conventional lenses, especially when coupled to optical fibers.

Furthermore, a double-clad fiber 39 is provided in the housing 28. This multi-clad fiber, in particular double-clad fiber, is a type of coaxial fiber and in the present example forms a first conduction means which is configured to transmit electromagnetic waves or beams between the illumination device to increase contrast 40 (cf. FIG. 1), which in the present case forms an observation area 41 (cf. FIG. 3a, right-hand side) in the manner of a spot on the tissue surface, in particular to increase contrast, for illumination, and a distal end of the endoscope.

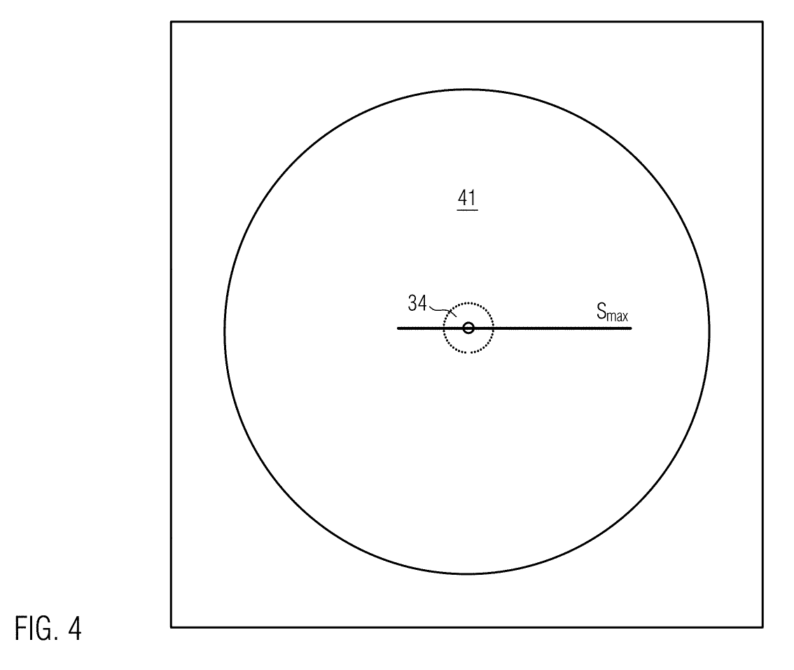

Furthermore, the double-clad fiber in the present example has a second conduction means configured to transmit electromagnetic waves or beams between the therapy device 42 (cf. FIG. 1) for the treatment of a therapy area 43 (cf. FIG. 4) and the distal end 5 of the endoscope 2. The therapy area 43 is formed in the same way as a spot on the tissue surface.

The double-clad fiber 39 contains a fiber core or a core fiber through which the therapy beam 44 is guided, which forms the therapy area 43 as a spot on the tissue surface, and a fiber cladding or cladding fiber. The illumination beam 45, which forms the observation area 41 as a spot on the tissue surface, is guided through the fiber core and the first fiber cladding.

A so-called double-clad fiber (DCF) can be a fiber, in particular glass fiber, which is composed of a core glass or core material (fiber core) and at least two cladding glasses or cladding materials (first fiber cladding and second fiber cladding), the inner cladding glass (first fiber cladding), which encloses the core glass, and the outer cladding glass (second fiber cladding), which encloses the inner cladding glass.

For example, all three glasses have different refractive indices. The core glass corresponds e.g. to that of the single mode fiber. For example, the inner cladding glass has a lower refractive index than the outer cladding glass.

The electromagnetic wave for lighting or the illumination beam can have a homogeneous angular distribution for the purpose of homogeneous lighting/illumination and can be guided in the fiber core and/or in the inner cladding glass and can be reflected by the outer fiber cladding, while the therapy beam or the electromagnetic waves for therapy, for example, is guided exclusively in the core, i.e. reflected by the first fiber cladding. As a synonym for Multi-clad Fiber, the term “Mehrmantelfaser” is also used, or as a synonym for the term Dual-clad fiber, the term “Zweimantelfaser” fiber is also used. As far as the term Multi-clad fiber is used, more than two light wave or electromagnetic wave conducting claddings are provided.

By integrating the first and second conduction means in a dual-clad or multi-clad fiber or also in a common fiber bundle, further miniaturization can be achieved and it is also ensured that the therapy area is centrally located in the lighting area. Preferred acceptance angles of the fiber core for the therapy beam are 3-9°, 4-8° and 5-6°. Preferred acceptance angles of the cladding core are 20-60°, 30-50° or 40°. The acceptance angle is the maximum angle at which light can be coupled into the fiber. The acceptance angle is given as the half-angle relative to the central axis of the light guide or the fiber, which also applies to the other acceptance angles given in this application.

A further possibility for cost savings and miniaturization can be the coupling into a single-clad fiber with a very high acceptance angle in such a way that the electromagnetic radiation of the illumination source is coupled strongly divergently with a homogeneous angular distribution, but the therapy source is coupled slightly divergently into the same single-clad fiber. The preferred acceptance angle is 20-60°, 30-50° or preferably 40°.

The illumination beam 45 generates the observation area 41 in a field of view 46 of the endoscope whose size is determined by the camera functionality. The observation area 41, for example, is slightly smaller than the field of view 46.

An area larger than that of the field of view 46 is illuminated by the cold light lamp 19, which thus serves as the illumination device for illumination and is referred to as the second illumination device, in contrast to the illumination device for contrast enhancement 40, which in this context is referred to as the first illumination device.

The area larger than that of the field of view is referred to as the illumination area and is generated, for example, by the field of view illumination fibers 14, 15, which is an example of a fourth and fifth conduction means.

The size of the field of view 46 is determined by the objective 13, the lens or the CCD chip on the front surface 4 of the endoscope.

The illumination device in the claims can be formed by the illumination device for floodlightning the observation area, or by the illumination device for increasing the contrast 40. For example, the illumination device for floodlightning the observation area can be integrated in the tube, and/or the illumination device for increasing the contrast 40 can be integrated in the insert 25.

FIG. 2b shows the corresponding areas. The large circle represents the field of view 46. Within field of view 46, a tumor 47 is schematically depicted in the tissue. The observation area 41 generated by the illumination beam 45 is directed towards the tissue in such a way that part of the tumor 47 lies within the observation area 41. In the example, the therapy area 43, which is generated by the therapy beam 44, is centrally located within the observation area. The therapy beam 44 has a certain wavelength which is necessary for the therapy of the tumor, in particular for destroying the same or for the therapy of any other tissue.

In contrast to the illumination beam, the therapy laser, for example, has a different wavelength and/or a different intensity so that the tumor or the material to be treated can be treated with it, in particular vaporized, resected or coagulated.

Preferred wavelengths of the therapy beam are 400 nm, 450 nm, 532 nm, 1470 nm, 1900 nm, 2200 nm, 3000 nm, 10,600 nm. These values can each form an upper or lower limit of a preferred range. Preferred intensities of the therapy beam are: 1 W, 5 W, 10 W, 20 W, 50 W, 100 W and 200 W. These values can form an upper or lower limit of a preferred range.

During therapy, an incision can be made around the tumor and/or tumor 47 can be destroyed/vaporized or degenerated/coagulated so that the tumor tissue dies. Due to the movement of the endoscope, for example over the Bowden cables, the observation area 41 can move together with the therapy area 43 over the tissue surface and thus degenerate the tumor bit by bit in a scanning movement as an example for an object to be treated.

During this therapy, depth information of at least the therapy area 43 and the observation area 41 is obtained according to the present invention using optical coherence tomography.

An image of a one-dimensional scan taken by optical coherence tomography is shown in FIG. 3b, for example. The dimensionality of the scan refers to the movement of the beam (1-dimensional) and not to the tissue image (2-dimensional).

Via the movable mirror 34, the optical coherence tomography beam 48 can be moved at least along the line S. shown in FIG. 3 within the observation range. In this example a one-dimensional scan takes place. Here the optical coherence tomography beam is directed to the corresponding surface positions and the reflected information is used to generate the optical coherence tomography image from FIG. 3b.

The Y-axis with the designation A-Scan, in FIG. 3b corresponds to the depth direction of the tissue, with the tissue surface visible at the top of the Figure and various deeper tissue layers below. The X-axis with the designation B-Scan, in FIG. 3b corresponds to the direction of the line S. in FIG. 3a.

If a tumor or other object to be treated is treated with the therapy beam, a change in the optical coherence tomography image can be detected directly and thus precise information about the success of the therapy during therapy is possible.

The term “at least one-dimensional” means that a two- or multi-dimensional screening or a two- or multi-dimensional scan is not excluded. In addition, the mirror can also be positioned in such a way that a two-dimensional scan and thus a three-dimensional image of the surface with the depth information is obtained in addition to the one-dimensional scanning A two-dimensional scan provides a three-dimensional image in relation to the area and depth of the observation area.

For this purpose, for example, the movable mirror can be guided through the observation area 41 in meander-like form by means of the control unit 21.

The detected OCT-radiation as well as the reflected or transmitted illumination radiation can be collected by the respective conduction means and transmitted to the corresponding device for the evaluation of the information.

These two images, the visual image or the field of view 46 and the OCT image can be superimposed simultaneously on a visualization device as shown in FIG. 1, displayed in different screen areas of the same screen, or several screens or visualization devices can be provided, which the treating practitioner sees in front of him during observation and therapy. The practitioner therefore sees an image of an illuminated area as well as at least the image obtained by optical coherence tomography and can thus at least gain the depth information in the point being treated.

In the example from FIG. 3a, the optical coherence tomography fiber 36 is provided a-centrically to a central axis Z and the distal end of the optical coherence tomography fiber 36 ends inside the housing 28. The optical coherence tomography beam 48 coupled from the distal end of the optical coherence tomography fiber 36 is deflected in the prism 35 to the movable mirror 34. The prism is an example of a deflecting device which is configured in such a way that the electromagnetic waves of the optical coherence tomography device 37 can be transmitted via it to the movable mirror 34.

The movable mirror 34 can be composed of one MEMS 59, which is vapor-deposited on its front side by means of a reflective layer 60, the side facing the deflector/prism 35. This movable mirror 34 can, for example, be moved by electrostatic actuators, which are not shown in the Figures. Especially small and fast scanner chips (MEMS) with dimensions <2 mm are deflected in resonance mode. The MEMS scanner can be provided with a power supply, e.g. a cable, which looks out at the proximal end of the endoscope 2 and is coupled to a corresponding control device for controlling the minor 34, which is provided for example in the base station 1.

In the present case, the movable minor itself is arranged obliquely so that the normal to the minor surface is oblique to the central axis Z of the housing 28 and thus also oblique to the optical coherence tomography beam 48 coupled out of the optical coherence tomography fiber 36. Through the oblique position of the minor surface and the central axis of the housing, it is achieved that the optical coherence tomography beam 48, as shown in FIG. 3a, is coupled out of the housing frontally at an angle Ay to the central axis Z.

For this purpose, an opening 61 is provided on the front surface 31 of the housing (see FIG. 2a). In the present case, opening 61 has an elongated oval design. This makes it possible to perform a one-dimensional scan transverse to the longitudinal direction L of the housing 28. Thus, the housing forms a holder for the minor and/or the corresponding conduction means.

As shown in FIG. 2a, the housing 28 can have a recess 62 on one circumferential surface. Through the recess 62, the MEMS 59 with the movable mirror 34 can be inserted into the housing 28 if the housing is provided as a one-piece element.

Within the housing at least one cavity can be provided, so that a certain mobility of the minor 34 is given for the scanning process.

The oblique irradiation of the optical coherence tomography beam 48 makes it possible to have a area where the optical coherence tomography beam 48 overlaps with the therapy beam 44 on the surface to be examined and treated. Depending on the distance from the tissue surface to the distal end of the endoscope, the angle between the coherence tomography beam 48 and the therapy beam 44 can be adjusted via the movable minor so that on the sample surface at least in one position of the optical coherence tomography beam 48 there is a cut between this and the therapy beam 44. This makes it possible to simultaneously receive the depth information during therapy in the therapy area.

FIG. 1 shows schematically that the first conduction means, second conduction means and third conduction means are coupled to the illumination device 40 or the therapy device 42 or the optical coherence tomography device 37 in the base station 1 via corresponding connectors. The illumination device 40, the therapy device 42 and the optical coherence tomography device 37 can, as described in detail above, also be provided as individual modules separately from base station 1 or can also be constructed as a combined module consisting of illumination device, therapy device and optical coherence tomography device.

Each of the aforementioned units, illumination unit for illumination, illumination unit for increasing contrast, camera unit, therapy device, optical coherence tomography device, Bowden cable control unit, can be provided as modular individual systems or in any combination with each other as combined subsystems.

For cost reasons, it is often preferable to use previously mentioned units that are already available in the practice and to purchase only those units whose functionality is not yet available.

Thus the example of the uniform base station is to be understood only as an example and does not limit the possible modular structure.

These systems can, for example, be controlled by a common control unit integrated in a PC, or by individual control modules that can communicate with each other for coordination purposes.

In particular, it is preferred that an application program, in addition to the program used to control the conventional device on the endoscopy system, be installed on the PC to ensure the additional functionality achieved by the insert or the combination of optical coherence tomography.

In the embodiments it has been described that both the first, second, and third conduction means are provided inside the housing or inside the insert which is part of the endoscope.

However, this is not necessarily the case.

It is quite sufficient that the endoscope has such first, second and third conduction means as described in claim 1. The conduction means may in some way be associated with the tube. For example, they may be at least partially enclosed by the tube. If the insert is inserted in the working channel or another free channel of the tube, it is also enclosed by the tube.

It is also possible that the insert is already inserted into a corresponding channel of the tube in the factory and is held captive in the channel Captive means that the insert can have a certain degree of mobility and/or rotatability in the longitudinal direction, but cannot be pulled out of the channel and will not be damaged.

In particular, as described here, all three conduction means can be combined in this housing or the insert.

Any known endoscope should be considered as an endoscope. In particular, in the case of the particularly small working channels of cystoscopes used for catheterisation of the urinary tract, it has been possible for the first time to offer such a small insert which can be inserted in the working channel

It has turned out that the housing can have a diameter in particular in the range of ≤mm. The working channel of the cystoscope can have a diameter in the range of ≤2.4 mm.

Preferably, the insert can be inserted into the working channel so that the distal end of the housing protrudes between 0, 1, 2, 3 and 4 mm from the working channel These specified values can be combined as upper or lower limits in any way.

The length of the one-dimensional OCT scan line (B scan) is preferably between 1 and 12 mm, 2 and 10 mm, 4 and 8 mm, in particular 6 mm. These values can be combined with each other as upper or lower limits of any kind.

The diameter of the observation area is preferably between 5 and 20 mm, 6 and 15 mm, 8 and 12 mm, in particular 10 mm. These values may each be combined as upper or lower limits in any way.

The diameter of the therapy area is preferably between 100 and 4000 μm, in particular 200 and 2000 μm, preferably 1000 μm. These indicated values can be combined as upper or lower limits in any way.

The diameter of the observation area or therapy area can be determined by the opening angle of the corresponding conduction means and the distance to the observation area for the corresponding illumination beam or therapy beam.

Accordingly, the fiber cladding or the cladding fiber and/or the fiber core or the core fiber can be designed in such a way that these fibers have different opening angles, so that the corresponding illumination or therapy beam is widened differently.

FIG. 4 schematically shows the combination of the observation area 41, the therapy area 43 and the scan line Smₐx of the therapy beam 44.

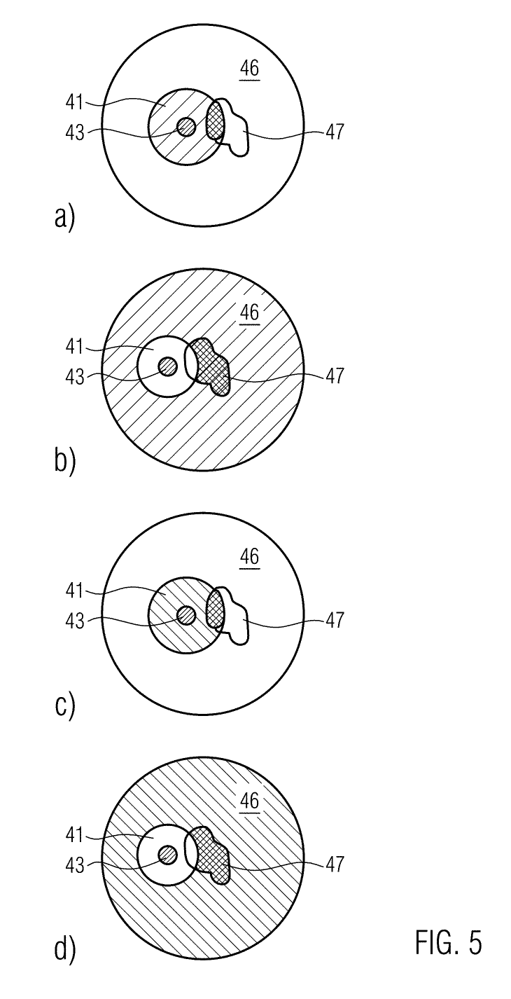

FIGS. 5a to d show various possible combinations of the illumination of the field of view 46 with the second illumination device, the illumination of the observation area 41 with the first illumination device, whereby the therapy area 43 can be seen centrally in the observation area 41.

The OCT scan or OCT beam is not displayed. The B scan of the OCT beam covers the therapy area in the center of the tissue plane and at most to the end of the illumination area.

FIGS. 5a, b, c, d show the tumor 47 within the field of view 46.

In the example in FIG. 5a, the illumination of the field of view 46 is an illumination with the cold light lamp 19 shown in FIG. 1. Any white light generation unit can be used for this purpose.

The observation area 41 is generated by means of the illumination device to increase the contrast 40. For example, a certain wavelength is used which interacts with a photosensitizer and thus increases the contrast. This is why, for example, the tumor is better visible in the observation area. FIG. 5a therefore uses a PDD system for illumination.

FIG. 5b shows the reverse situation in which the illumination of the field of view 46 is carried out by means of the PDD system. Observation area 41 is generated with white light. This allows the interaction of the therapy beam with the tissue to be better observed and the endpoint of the therapy to be better determined.

FIGS. 5a and 5b therefore use a PDD system and a white light system as the first and second lighting devices.

In FIGS. 5c and d, on the other hand, a so-called NBI system is used instead of the PDD system.

First, observation area 41, as shown in FIG. 5c, can be generated by the NBI system. As described above, for example, two different radiation wavelengths are used to increase contrast.

For example, the field of view 46 shown in FIG. 5c is illuminated with white light.

FIG. 5d shows the reverse situation, where field of view 46 is illuminated by an NBI system and observation area 41 is generated by a white light source.

The size of the observation area and the therapy area in the Figures are determined by the embodiment of the first and second conduction means respectively.

The size of the field of view 46, on the other hand, is determined, for example, by the objective 13.

If the therapy area lies within the observation area, which is advantageously the case, then parts of the observation area are also imaged with that of an optical coherence tomography device.

### REFERENCE NUMERAL LIST

1 base station
2 endoscope
3 tube
4 front surface of the endoscope
5 distal end of endoscope
6 working channel
7 second channel
8 third channel
9 camera channel
10 visible area
11 fifth channel
12 sixth channel
13 lens
14, 15 field of view illumination fiber
17 multi-pole plug
18 Bowden cable control unit
19 cold light lamp/illumination unit for floodlightning the observation area
20 camera unit
21 control unit
22 input station
23 visualization device/screen
24 insert
24a, 24b, 24c insert plug
25a free opening
25b lens
25 side opening
26 front opening of the working channel
27 distal end of insert
28 housing
29 first fiber
30 second fiber
31 front surface of the housing
32 circumferential surface
33 rear surface
34 movable mirror
35 prism/deflecting device
36 optical coherence tomography fiber
37 optical coherence tomography device
39 double-clad fiber/coaxial fiber
40 illumination unit to increase contrast
41 observation area
42 therapy device
43 therapy area
44 therapy beam
45 light beam
46 field of view
47 tumor
48 optical coherence tomography beam
59 MEMS
60 reflective layer
61 opening on the front surface of the housing
62 recess
63 protective cover
64 Grin lens
O tissue surface
Z central axis of the housing
L longitudinal direction
Q transverse direction

1. An endoscope, comprising
a first conduction means configured to transmit electromagnetic waves between an illumination means for illuminating an observation area and a distal end of the endoscope; and
a second conduction means configured to transmit electromagnetic waves between a therapy device for treating a therapy area within the observation area and the distal end of the endoscope, characterized in that
a third conduction means is provided which is configured to transmit electromagnetic waves between an optical coherence tomography device by means of which depth information about said area is obtained during treatment of the therapy area and the distal end of the endoscope.

2. The endoscope according to claim 1, characterized in that at least two, preferably three, of the following distal ends are integrally provided in a tube of the endoscope: a distal end of the first conduction means, a distal end of the second conduction means, a distal end of the third conduction means.

3. The endoscope according to claim 1, characterized in that at least two, preferably three, of the following distal ends are provided within a housing: a distal end of the first conduction means, a distal end of the second conduction means, a distal end of the third conduction means, an outer diameter of the housing being configured such that it can be inserted into a working channel of the endoscope designed as a cystoscope.

4. The endoscope according to claim 1, characterized in that the first and second conduction means are formed by a multi-clad, in particular double-clad fiber, having a fiber core and at least two cladding glasses, so that these form a first fiber cladding and a second fiber cladding, wherein the first fiber cladding encloses the fiber core and the second fiber cladding encloses the first fiber cladding.

5. The endoscope according to claim 4, characterized in that the fiber core and the at least two cladding glasses each have different refractive indices, and in particular the first fiber cladding has a lower refractive index than the second fiber cladding.

6. The endoscope according to claim 5, characterized in that the first conduction means is formed by the first fiber cladding and/or the fiber core, and in that the second conduction means is formed by the fiber core.

7. The endoscope according to claim 4, characterized in that the fiber core has an acceptance angle of 3-9°, and/or in that the first fiber cladding has an acceptance angle of 20-60°.

8. The endoscope according to claim 1, characterized in that a common single-clad fiber is provided as first and second conduction means, and in that the illumination device and the therapy device are each configured in such a way that the electromagnetic waves of the illumination device are coupled divergently into the single-clad fiber with a homogeneous angular distribution, and in that the electromagnetic waves of the therapy device are coupled into the single-clad fiber with an opening angle of less than 9°.

9. The endoscope according to claim 8, characterized in that the single-clad fiber has an acceptance angle of 20-60°.

10. The endoscope according to claim 1, characterized in that the third conduction means has at its distal end within the housing a movable mirror and a deflecting device which are configured in such a way, in that the electromagnetic waves of the optical coherence tomography device can be transmitted via the deflecting device to the movable mirror, and can be irradiated by the movable mirror through a first opening in a front surface of the housing at least into the observation area.

11. The endoscope according to claim 10, characterized in that the movable mirror is configured in such a way that, during the treatment of the therapy area, it carries out an at least one-dimensional scan through the therapy area.

12. The endoscope according to claim 11, characterized in that the movable mirror is configured in such a way that during the treatment of the therapy area it carries out an at least one-dimensional scan through the housing at a distance of 5-20 mm from the distal end of the housing, which scan completely encloses the therapy area and the observation area in one direction.

13. The endoscope according to claim 1, characterized in that the third conduction means has a fiber whose distal end is provided acentrically in the interior of the housing and from which the electromagnetic waves of the optical coherence tomography device can be coupled into the deflecting device.

14. The endoscope according to claim 10, characterized in that the movable mirror is arranged obliquely to a central axis of the housing so that the electromagnetic waves which can be radiated through the first opening in the front surface of the housing at least onto the therapy area can be radiated at a predetermined angle to the central axis of the housing.

15. The endoscope according to claim 14, characterized in that the therapy beam formed by the electromagnetic waves for the treatment of a therapy area intersects the illumination beam formed by the electromagnetic waves for the illumination of an observation area and the illumination beam formed by the electromagnetic waves of the therapy area at a distance of between 5 and 15 mm from an outcoupling end of the first and conduction means.

16. The endoscope according to claim 4, characterized in that the multi-clad, in particular double-clad fiber, is provided acentrically inside the housing and the electromagnetic waves coupled out of the housing can be irradiated parallel to the central axis of the housing onto the observation area and/or therapy area.

17. The endoscope according to claim 4, characterized in that a distal end of the multi-clad, in particular double-clad, fiber projects beyond the front surface of the housing.

18. The endoscope according to claim 4, characterized in that the multi-clad, in particular double-clad fiber is designed in such a way that the therapy area lies centrally within the observation area.

19. The endoscope according to claims 3, characterized in that the housing has a diameter in the range of 2 mm or smaller.

20. The endoscope according to claim 1, characterized in that the illumination device comprises at least one of the following systems: NBI system, RGB system, PDD system, white light system.

21. The endoscope according to claim 20, characterized in that the RGB system comprises a red laser operating in the wavelength range from 600 nm to 699 nm, a green laser operating in the wavelength range from 500 nm to 599 nm, and a blue laser operating in the wavelength range from 400 nm to 499 nm.

22. The endoscope according to claim 1, characterized in that the therapy device has a therapy laser which operates in the following wavelength ranges 400 nm to 10,600 nm, in particular 400 nm to 3000 nm, preferably 400 nm, to 2200 nm, in particular preferably at 440-460 nm.

23. The endoscope according to claim 22, characterized in that a therapy beam with an opening angle of less than 9°, preferably less than 6°, can be decoupled from the therapy device.

24. The endoscope according to claim 21, characterized in that a laser beam with an opening angle of 20° or more can be decoupled from the RGB system.

25. The endoscope according to claim 21, characterized in that the therapy beam coupled out of the RGB system and the therapy beam coupled out of the therapy device can be coupled into the single-clad fiber, or the dual-clad fiber, or the multi-clad fiber in such a way that the beam of therapy beam and illumination beam coupled out of the corresponding fiber has a homogeneous beam profile at a distance of between 5 and 15 mm from the decoupling location.

26. The endoscope according to claim 25, characterized in that the single-clad fiber, or the dual-clad fiber, or the multi-clad fiber, which form the first and second conduction means, has a larger acceptance angle than the opening angle of the RGB system and/or the therapy device.

27. The endoscope according to claim 25, characterized in that the single-clad fiber, or the dual-clad fiber, or the multi-clad fiber forming the first and second conduction means, has a length of between 0.5 m and 10 m.

28. The endoscope according to claim 25, characterized in that a laser beam with an opening angle of more than 20° can be decoupled from the first and/or conduction means.

29. A system for simultaneous observation of an observation area and treatment of a therapy area within the observation area comprising:
an endoscope;
an illumination device for illuminating the observation area;
an therapy device for treating the therapy area within the observation area;
an optical coherence tomography device, by means of which depth information about this area can be obtained during the treatment of the therapy area; and
a control device by means of which the illumination device, the therapy device and the optical coherence tomography device can be controlled.

30. An insert which is adapted to be fitted into a working channel of an endoscope, wherein the insert has a housing and is arranged inside the housing,
a first conduction means configured to transmit electromagnetic waves between an illumination device for illuminating an observation area and a distal end of the endoscope,
a second conduction means configured to transmit electromagnetic waves between a therapy device for treating a therapy area within observation area and the distal end of the endoscope,
and a third conduction means configured to transmit electromagnetic waves between an optical coherence tomography device by means of which depth information about said area can be obtained during treatment of the therapy area and the distal end of the endoscope.
